(19) United States
(12) Patent Application Publication
(10) Pub. No.: US 2011/0224606 A1
(43) Pub. Date: Sep. 15, 2011

(54) METHOD AND APPARATUS FOR REMOTE ISCHEMIC CONDITIONING DURING REVASCULARIZATION

(76) Inventors: SHIBAJI SHOME, ARDEN HILLS, MN (US); ERIC A. MOKELKE, WHITE BEAR LAKE, MN (US); ROGER HASTINGS, MAPLE GROVE, MN (US); ALLAN C. SHUROS, ST. PAUL, MN (US); JAMES A. ESLER, COON RAPIDS, MN (US)

(21) Appl. No.: 13/036,941

(22) Filed: Feb. 28, 2011

Related U.S. Application Data
(60) Provisional application No. 61/312,521, filed on Mar. 10, 2010.

(51) Int. Cl. A61M 25/10

(52) U.S. Cl. 604/96.01

(57) ABSTRACT

Remote ischemic conditioning is applied during a revascularization procedure to prevent and/or reduce myocardial injury associated with myocardial infarction (MI) and the revascularization procedure such as percutaneous transluminal coronary angioplasty (PTCA). A percutaneous transluminal vascular intervention (PTVI) device used for the revascularization procedure, such as an introducer sheath or a guide catheter, includes an adjustable balloon to be positioned at a vascular site remote from the heart. The remote ischemic conditioning is applied by inflating and deflating the adjustable balloon, thereby causing temporary ischemia in the vascular site to activate the patient's intrinsic cardioprotective mechanism.

### CLAIM OF PRIORITY

This application claims the benefit of priority under 35 U.S.C. §119(e) of U.S. Provisional Patent Application Ser. No. 61/312,521, filed on Mar. 10, 2010, which is herein incorporated by reference in its entirety.

### TECHNICAL FIELD

This document relates generally to medical devices and particularly to percutaneous transluminal vascular intervention (PTVI) devices that provide for protection of a heart against injuries associated with cardiac ischemia by applying ischemic conditioning to one or more vascular sites remote from the heart during a revascularization procedure.

### BACKGROUND

The heart is the center of a person's circulatory system. It includes an electro-mechanical system performing two major pumping functions. The left portions of the heart draw oxygenated blood from the lungs and pump it to the organs of the body to provide the organs with their metabolic needs for oxygen. The right portions of the heart draw deoxygenated blood from the body organs and pump it to the lungs where the blood gets oxygenated. These pumping functions result from contractions of the myocardium (cardiac muscles). In a normal heart, the sinoatrial node, the heart's natural pacemaker, generates electrical impulses, called action potentials, that propagate through an electrical conduction system to various regions of the heart to excite the myocardial tissues of these regions. Coordinated delays in the propagations of the action potentials in a normal electrical conduction system cause the various portions of the heart to contract in synchrony to result in efficient pumping functions. A blocked or otherwise abnormal electrical conduction and/or deteriorated myocardial tissue cause dyssynchronous contraction of the heart, resulting in poor hemodynamic performance, including a diminished blood supply to the heart and the rest of the body. The condition in which the heart fails to pump enough blood to meet the body's metabolic needs is known as heart failure.

Myocardial infarction (MI) is the necrosis of portions of the myocardial tissue resulted from cardiac ischemia, a condition in which the myocardium is deprived of adequate oxygen supply and metabolite removal due to an interruption in blood supply caused by an occlusion of a blood vessel such as a coronary artery. The necrotic tissue, known as infarcted tissue, loses the contractile properties of the normal, healthy myocardial tissue. Consequently, the overall contractility of the myocardium is weakened, resulting in an impaired hemodynamic performance. Following an MI, cardiac remodeling starts with expansion of the region of infarcted tissue and progresses to a chronic, global expansion in the size and change in the shape of the entire left ventricle. The consequences include a further impaired hemodynamic performance and a significantly increased risk of developing heart failure.

When a blood vessel such as the coronary artery is partially or completely occluded, a revascularization procedure such as percutaneous transluminal coronary angioplasty (PTCA) can be performed to reopen the occluded blood vessel. However, the revascularization procedure itself involves a temporary occlusion of the coronary artery. During a period of occlusion of an artery, stagnant blood may clot in the vascular bed distal of the occlusion. Starved of oxygen, the myocardium in the ischemic region undergoes anaerobic metabolism, which generates lactic acid and other constituents that lower the pH of the tissue and puts the myocardium into a state of hibernation in which the cells are alive but not contracting. Reperfusion that follows the reopening of the occluded blood vessel is also known to cause cardiac injury, known as reperfusion injury. The hibernating myocardium is in an oxygen deprived and weakened state, and may be further damaged or killed by the sudden onrush or fresh blood following reperfusion. The rapid normalization of pH and rush of calcium into the cells may cause a sudden and forceful initial contraction that can damage the cells. Fresh blood may poorly perfuse tissue fed by clotted vessels, while it rushes into other areas causing edema that further constricts vessels. Reactive oxygen species that are formed during reperfusion can damage cells if they are not quickly washed away. In addition, plaques dislodged and displaced by the revascularization procedure may enter small blood vessels branching from the blood vessel in which the revascularization is performed, causing occlusion of these small blood vessels. The revascularization procedure may also cause distal embolization, i.e., obstruction of the artery caused by the plaque dislodged during the procedure. Therefore, there is a need for minimizing cardiac injury associated with MI and the subsequent revascularization procedure.

### SUMMARY

Remote ischemic conditioning is applied during a revascularization procedure to prevent and/or reduce myocardial injury associated with myocardial infarction (MI) and the revascularization procedure such as percutaneous transluminal coronary angioplasty (PTCA). A percutaneous transluminal vascular intervention (PTVI) device used for the revascularization procedure, such as an introducer sheath or a guide catheter, includes an adjustable balloon to be positioned at a vascular site remote from the heart. The remote ischemic conditioning is applied by inflating and deflating the adjustable balloon, thereby causing temporary ischemia in the vascular site to activate the patient's intrinsic cardioprotective mechanism.

In one embodiment, a PTVI device includes a proximal end, a distal end configured to be placed in one or more arteries, and an elongate tubular shaft coupled between the proximal end and the distal end. A lumen extends within the elongate tubular shaft and includes a proximal opening at the proximal end of the PTVI device and distal opening at the distal end of the PTVI device. The lumen is configured to accommodate a portion of an angioplasty catheter and allow the distal end of the angioplasty catheter to enter the proximal opening of the lumen and exit from the distal opening of the lumen. An adjustable balloon is incorporated onto the elongate tubular shaft and configured to occlude a portion of the one or more arteries. The balloon includes a chamber having an adjustable volume. An elongate balloon adjustment tube is coupled to the adjustable balloon and includes a fluid passageway in fluid communication with the chamber to allow inflation and deflation of the adjustable balloon.

In one embodiment, a method for cardioprotection during revascularization of a coronary artery is provided. A portion of a PTVI device is inserted into one or more arteries. The PVTI device includes an adjustable balloon. A portion of an angioplasty catheter is inserted into a lumen of the PTVI device. A distal end of the angioplasty catheter is advanced through the PTVI device into the coronary artery. A portion of the one or more arteries is temporarily occluded by inflating and deflating the adjustable balloon. The occlusion is timed using parameters selected to create a physiologic stress that is sufficient to trigger an intrinsic myocardial protective mechanism.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof. The scope of the present invention is defined by the appended claims and their legal equivalents.

### BRIEF DESCRIPTION OF THE DRAWINGS

The drawings illustrate generally, by way of example, various embodiments discussed in the present document. The drawings are for illustrative purposes only and may not be to scale.

FIG. 1 is an illustration of an embodiment of a system providing for remote ischemic conditioning during revascularization and portions of an environment in which the system is used.

FIG. 2 is a flow chart illustrating an embodiment of a method for applying remote ischemic conditioning therapy during revascularization.

FIG. 3 is a timing diagram illustrating an embodiment of a remote ischemic conditioning sequence.

FIG. 4 is an illustration of an embodiment of a PTVI device with an adjustable balloon for remote ischemic conditioning.

FIG. 5 is an illustration of another embodiment of a PTVI device with an adjustable balloon for remote ischemic conditioning.

FIG. 6 is an illustration of another embodiment of a PTVI device with an adjustable balloon for remote ischemic conditioning.

FIG. 7 is an illustration of an embodiment of an introducer sheath with an adjustable balloon for remote ischemic conditioning.

FIG. 8 is an illustration of an embodiment of a guide catheter with an adjustable balloon for remote ischemic conditioning.

FIG. 9 is an illustration of an embodiment using the introducer sheath and/or the guide catheter during a revascularization procedure.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention. The following detailed description provides examples, and the scope of the present invention is defined by the appended claims and their legal equivalents.

It should be noted that references to “an”, “one”, or “various” embodiments in this disclosure are not necessarily to the same embodiment, and such references contemplate more than one embodiment.

In this document, “revascularization” includes reopening of a completely or partially occluded blood vessel using percutaneous transluminal vascular intervention (PTVI) procedure, such as a percutaneous transluminal coronary angioplasty (PTCA) procedure performed in response to cardiac ischemia or myocardial infarction (MI), using PTVI devices such as those discussed in this document.

In this document, when used in relation to a PTVI device, “distal” refers to the direction toward which the PTVI device is introduced into, and advanced through, one or more blood vessels in the patient's body, and “proximal” refers to the opposite direction. For example, a dilatation balloon catheter typically includes a dilatation balloon at or near its distal end. The distal end may be introduced into a femoral artery and advanced through arteries into a coronary artery. The proximal end of the dilatation balloon catheter remains outside of the body throughout the procedure. In this document, when used in relation to a PTVI device, “upstream” and “downstream” refer to locations relative to the direction of blood flow in a blood vessel after a portion of the PTVI device is positioned in that blood flow. Thus, assuming points A, B, and C are on a portion of a PTVI device placed in a blood vessel within which the blood flows toward the proximal direction of the PTVI device and flows from A to B and then to C, then location A is “distal to location B” or “upstream to location B’, and location C is “proximal to location B” or “downstream to location B”.

This document discusses a system for applying remote ischemic conditioning therapy to a patient receiving a revascularization procedure through one or more PTVI devices used in the revascularization procedure. Remote conditioning includes occlusion of a major artery feeding a vascular bed that is remote from the patient's heart. Remote conditioning may be performed before and during the revascularization procedure, and may consist of one or more repeated occlusions. In one example, remote conditioning is performed by inflation of a blood pressure cuff on an arm or leg of a patient suffering from an acute myocardial infarction. Occlusion of the remote tissue generates compounds locally and through the body's neuro-hormonal feedback system, thereby protecting not only the remote ischemic tissue, but the ischemic tissue in the heart. However, because the use of a blood pressure cuff may not be a well controlled procedure, the degree of ischemia is difficult to monitor, especially in a leg. The present system is to be operated by a trained physician or other caregiver in the setting of an angioplasty procedure to deliver remote ischemic conditioning therapy by occlusion of a major artery using an adjustable device placed in that major artery.

According to the present subject matter, the remote ischemic conditioning therapy induces brief periods of ischemia at one or more vascular sites remote from the patient's heart to elicit physiological protective effect against myocardial damage associated with cardiac ischemia, including ischemic and reperfusion injuries. In various embodiments, one or more adjustable balloons are incorporated into the one or more PTVI devices for positioning in one or more peripheral vascular locations during the revascularization procedure. The remote ischemic conditioning therapy is applied by inflating and deflating the one or more adjustable balloons before, during, and/or after the temporary occlusion of the blood vessel that is to be reopened by the revascularization procedure, such as a coronary artery.

In various embodiments, one or more remote ischemic conditioning sequences are applied to the patient during the revascularization procedure. Each remote ischemic conditioning sequence includes alternating ischemic and non-ischemic periods. The alternating ischemic and non-ischemic periods are timed using parameters selected to create physiologic stress that is sufficient to trigger an intrinsic myocardial protective mechanism against myocardial injury. During each of the ischemic periods, the one or more adjustable balloons are inflated to temporarily occlude one or more blood vessels at the one or more vascular sites. During each of the non-ischemic periods, the one or more adjustable balloons are deflated.

While an introducer sheath and a guide catheter are specifically discussed in this document as examples of PTVI devices with balloons for remote ischemic conditioning, the present subject matter includes one or more adjustable balloons incorporated into any one or more PTVI devices used during revascularization. Each adjustable balloon is located on a PTVI device such that when the PTVI device is positioned in the patient's body, the adjustable balloon is positioned at a vascular site suitable for applying the remote ischemic conditioning therapy.

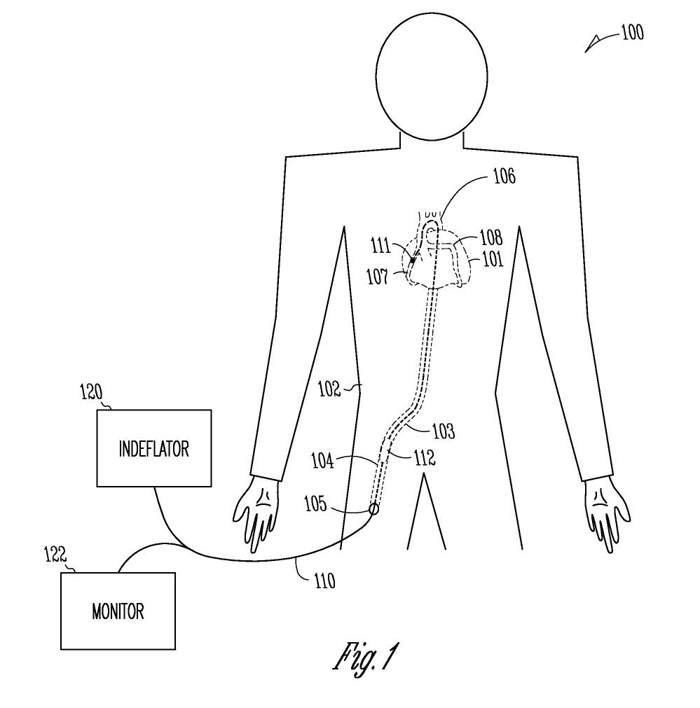

FIG. 1 is an illustration of an embodiment of a system 100 that provides for remote ischemic conditioning during revascularization and portions of an environment in which system 100 is used. System 100 includes a PTVI device assembly 110 connected to an indeflator 120 and optionally a monitor 122. PTVI device assembly 110 represents a combination of PTVI devices used during a revascularization procedure, such as a PTCA procedure performed in response to an MI that occurred in a heart 101.

In one embodiment, PTVI device assembly 110 includes PTVI devices used to perform the PTCA procedure. During the PTCA procedure, an opening 105 is made on a femoral artery 104 in a patient's body 102. The PTVI devices of PTVI device assembly 110 are each inserted into one or more arteries including femoral artery 104, an iliac artery 103, an aorta 106 and a right coronary artery 107, such that an angioplasty device 111 is advanced into right coronary artery 107. In another embodiment, PTVI device assembly 110 is similarly used but with angioplasty device 111 being advanced into a left coronary artery 108.

PTVI device assembly 110 includes an adjustable balloon 112 incorporated into one of the PTVI devices such that when PTVI device assembly 110 is in place for performing angioplasty in right coronary artery 107 or left coronary artery 108, adjustable balloon 112 is in a location suitable for applying remote ischemic conditioning. Indeflator 120 is used to inflate and deflate adjustable balloon 112 during a remote ischemic conditioning sequence. In one embodiment, indeflator 120 is manually operated according to a specified remote ischemic conditioning sequence. In another embodiment, indeflator 120 automatically controls the inflation and deflation of adjustable balloon 112 according to the specified remote ischemic conditioning sequence. In various embodiments, one or more remote ischemic conditioning sequences are initiated before, during, and/or after the angioplasty according to a therapy protocol.

In one embodiment, one or more sensors are incorporated into PTVI device assembly 110 to sense one or more signals such as blood pressure and/or flow, and monitor 122 processes the one or more sensed signals. In one embodiment, at least one signal is sensed to verify occlusion of a blood vessel by adjustable balloon 112.

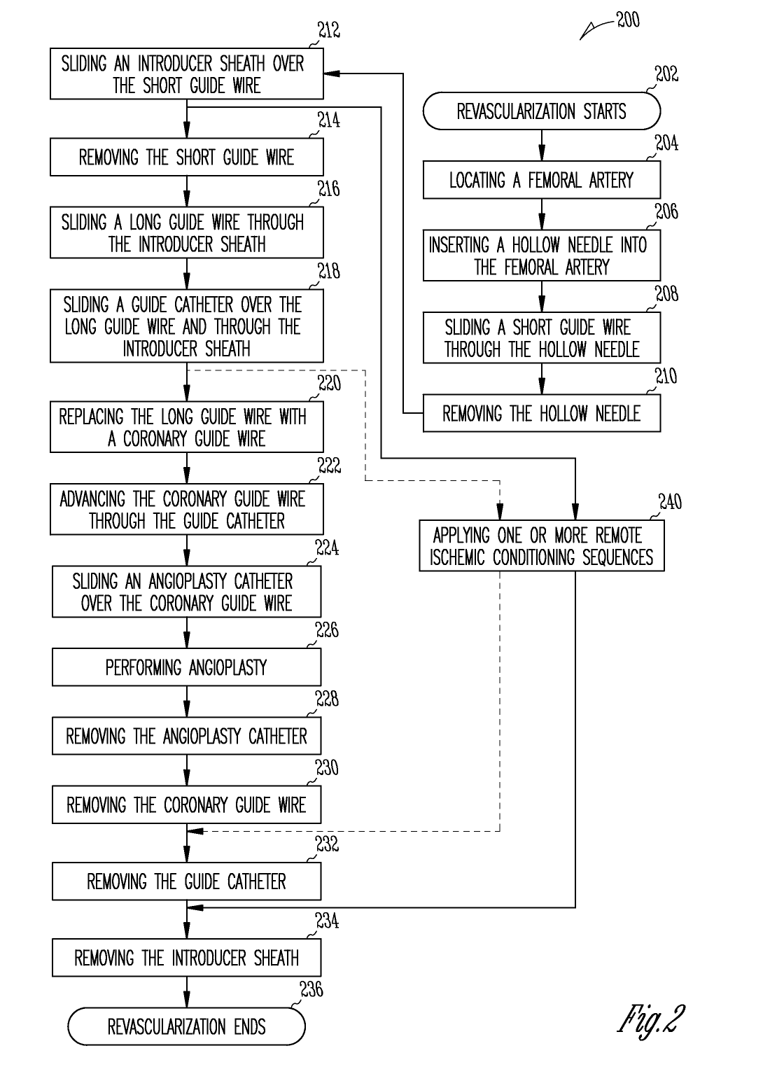

In one embodiment, for the purpose of the PTCA procedure, PTVI device assembly 110 includes, by way of example, and by way of limitation, PTVI devices selected from the following:
(i) A hallow needle to be inserted into the femoral artery;
(ii) Guide wires, such as guide wires selected from ChoICE® PT and PT Graphix™, PT2®, ChoICE®, Forté®, Platinum Plus™, and IQ® guide wire families (Boston Scientific Corporation, Natick, Mass., U.S.A.), including:
   a. A short guide wire to be introduced into and advanced within the femoral artery;
   b. A long guide wire to be introduced into and advanced through the aorta to the opening of a coronary artery, such as a guide wire having a 0.035-inch diameter; and
   c. A coronary guide wire to be introduced into and advanced through the aorta into the coronary artery, as a guide wire having a 0.014-inch diameter;
(iii) An introducer sheath, such as a Super Sheath™ introducer sheath (Boston Scientific Corporation), or an introducer sheath similar to the Super Sheath™ introducer sheath but including an adjustable balloon;
(iv) A guide catheter, such as a guide catheter selected from the Runway®, Mach 1®, and Wiseguide® guide catheters (Boston Scientific Corporation), or a guide catheter similar to one selected from the Runway®, Mach 1®, and Wiseguide® guide catheters but including an adjustable balloon; and
(v) Angioplasty catheters, including one or more of:
   a. A dilatation balloon catheter, such as a dilatation balloon catheter selected from the Apex®, Flextome® Cutting Balloon®, Maverick®, and Quantum™ Maverick® dilatation balloon catheters (Boston Scientific Corporation, Natick, Mass., U.S.A.);
   b. A stent delivery catheter, such as a stent delivery catheter selected from the Express²®, PROMUS®, TAXUS® Express²® Atom™, TAXUS® Express²® Coronary Stent System, TAXUS® Liberté®, TAXUS® Liberté®, and VeriFLEX™ stent delivery systems (Boston Scientific Corporation);
   c. An atherectomy device, such as an atherectomy catheter of the Rotablator® rotational atherectomy system (Boston Scientific Corporation); and
   d. An embolization protection device, such as an embolization protection catheter of the FilterWire EZ™ embolic protection system (Boston Scientific Corporation).
      As discussed below with reference to FIG. 2, PTVI device assembly 110 includes PTVI devices each being introduced into, and removed from, body 102 at different stages of the PTCA procedure. Thus, PTVI device assembly 110 includes different combinations of PTVI devices that are inserted into body 102 at different times. According to the present subject matter, at least one of the introducer sheath and the guide catheter includes adjustable balloon 112 for applying the remote ischemic conditioning therapy. In various embodiments, PTVI device assembly 110 may include PTVI devices selected from those listed above as well as additional PTVI devices not listed above, and one or more adjustable balloons are incorporated into one or more PTVI devices for placement in vascular locations suitable for applying the remote ischemic conditioning therapy.

FIG. 2 is a flow chart illustrating an embodiment of a method 200 for applying remote ischemic conditioning therapy during revascularization. By way of example, and not by way of limitation, PTVI device assembly 110, including the PTVI devices as discussed above, is used to perform method 200 as a PTCA procedure during which the remote ischemic conditioning therapy is applied.

At 202, performance of a revascularization procedure for a patient starts. In the illustrated embodiment, the revascularization procedure is a PTCA procedure. At 204, the femoral artery is located, for example by feeling for the pulses with fingers. At 206, the hollow needle is inserted into the femoral artery. At 208, the short guide wire is slid into the femoral artery through the hollow needle. At 210, the hollow needle is removed from the patient. At 212, the introducer sheath is slid into the femoral arteries over the short guide wire. At 214, the short guide wire is removed from the patient. At 216, the long guide wire is slid into the femoral artery through the introducer sheath and advanced through the aorta to the area of the coronary artery. At 218, the guide catheter is slid through the introducer sheath and over the long guide wire and advanced through the aorta until the distal end of the guide catheter engages the coronary artery at the opening of the coronary artery in the aorta. At 220, the long guide wire is replaced with the coronary guide wire, which is thinner than the long guide wire and has a diameter suitable for insertion into the coronary artery. At 222, the coronary guide wire is advanced through the guide catheter into the coronary artery to a target zone. The target zone includes a portion of the coronary artery that is to be opened by angioplasty. At 224, the angioplasty catheter is slid over the coronary guide wire and into the coronary artery. In one embodiment, steps 222 and 224 are performed together, i.e., the angioplasty catheter and coronary wire are advanced together to the coronary artery.

At 226, the angioplasty is performed. This includes, for example, dilating the target zone of the coronary artery by inflating the balloon on the dilatation balloon catheter. In another example, a stent is placed in the target zone of the coronary artery using the stent delivery catheter. In other examples, additional one or more steps such as coronary imaging, atherectomy, and/or embolization protection are also performed.

At 228, the angioplasty catheter is removed from the patient. At 230, the coronary guide wire is removed from the patent. At 232, the guide catheter is removed from the patient. At 234, the introducer sheath is removed from the patient. At 236, the performance of the revascularization procedure is concluded.

During the revascularization procedure, at 240, one or more remote ischemic conditioning sequences are applied after the PTVI device with the adjustable balloon is placed in a vascular site suitable for remote ischemic conditioning. The vascular site suitable for the remote ischemic conditioning is in one of the arteries through which the distal end of the angioplasty catheter is advanced into the coronary artery. The introducer sheath and the guide catheter remain in the arteries during most of the PTCA procedure. In one embodiment, the introducer sheath includes the adjustable balloon, and the one or more remote ischemic conditioning sequences are applied after the introducer sheath is slid into the femoral artery at 212 and before the introducer sheath is removed from the femoral artery at 234. In another embodiment, the guide catheter includes the adjustable balloon, and the one or more remote ischemic conditioning sequences are applied after the guide catheter is slid into the arteries with its distal end engaging the coronary artery at the opening of the coronary artery at 218 and before the guide catheter is removed at 232.

FIG. 3 is a timing diagram illustrating an embodiment of a remote ischemic conditioning sequence of the one or more remote ischemic conditioning sequences applied at 240. In various embodiments, the one or more remote ischemic conditioning sequences are each applied during the PTCA procedure as an ischemic preconditioning or periconditioning therapy (before an occlusion of the coronary artery begins during the angioplasty) or an ischemic postconditioning therapy (after the occlusion of the coronary artery begins during the angioplasty). In one embodiment, indeflator 120 is programmed to control the inflation and the deflation of adjustable balloon 112 according to the remote ischemic conditioning sequence. In one embodiment, indeflator 120 is programmed to start the remote ischemic conditioning sequence in response to a command entered by a physician or other caregiver participating in the PTCA procedure, and to automatically control the inflation and the deflation of adjustable balloon 112 according to the remote ischemic conditioning sequence.

The remote ischemic conditioning sequence includes a specified number (N) of cycles of alternating ischemic and non-ischemic periods. During each of the ischemic periods, the adjustable balloon is inflated to temporary occlude an artery (such as the femoral artery or the iliac artery). During each of the non-ischemic periods, the adjustable balloon is deflated. The number of cycles (N), the ischemic period, and the non-ischemic period are timed using parameters selected to create a physiologic stress in the patient that is sufficient to trigger an intrinsic myocardial protective mechanism against ischemic damage to the myocardial tissue. In one embodiment, the number of cycles (N) is programmable between approximately 1 and 10 cycles, with approximately 3 cycles being a specific example, the ischemic period is programmable between approximately 1 and 20 minutes, with approximately 4 minutes being a specific example, and the non-ischemic period is programmable between approximately 1 and 10 minutes, with approximately 4 minutes being a specific example. In the illustrated embodiment, it is not necessary to time the non-ischemic period of the last cycle (CYCLE N) because after the remote ischemic conditioning sequence is completed, the adjustable balloon is deflated (and then removed with the PTVI device onto which it is incorporated).

In one embodiment, a remote ischemic conditioning sequence is initiated before the reperfusion begins following the opening of the coronary artery by the angioplasty. In one embodiment, a remote ischemic conditioning sequence is initiated while the coronary artery is occluded by the angioplasty catheter. In various embodiments, the one or more remote ischemic conditioning sequences are each initiated by the physician or other caregiver when cardioprotection is needed.

In one embodiment, in which the introducer sheath includes the adjustable balloon, the remote ischemic conditioning sequence is applied with the adjustable balloon placed in the femoral artery. In another embodiment, in which the guide catheter includes the adjustable balloon, the remote ischemic conditioning sequence is applied with the adjustable balloon placed in the iliac artery. In one embodiment, the adjustable balloon is displaceable along the guide catheter. After the guide catheter is in place, the adjustable balloon is slid to a desirable site in the femoral artery, the iliac artery, or the descending aorta to apply the remote ischemic conditioning sequence. In one specific example, the adjustable balloon is slid to the descending aorta at a level above the iliac bifurcation to apply the remote ischemic conditioning sequence.

In one embodiment, blood flow and/or blood pressure are sensed at a vascular location upstream to the location where the adjustable balloon is placed during the application of the remote ischemic conditioning sequence. In one embodiment, blood flow and/or blood pressure are sensed at a vascular location downstream to the location where the adjustable balloon is placed during the application of the remote ischemic conditioning sequence. Such blood flow and/or pressure sensing upstream and/or downstream to the occlusion site provides for verification of the occlusion of the blood vessel by the adjustable balloon. In one embodiment, the pressure and/or flow are sensed using sensors incorporated onto the PTVI device onto which the adjustable balloon is incorporated.

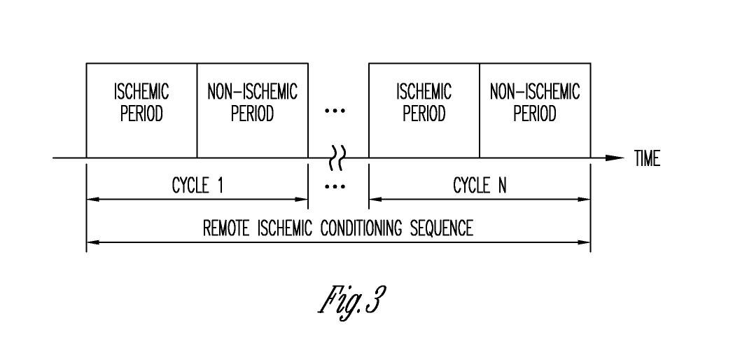

FIG. 4 is an illustration of an embodiment of a PTVI device 450 for remote ischemic conditioning. PTVI device 450 is part of PTVI device assembly 110 and includes a proximal end 452, a distal end 451, an elongate tubular shaft 453, a lumen 454, an adjustable balloon 456, and an elongate balloon adjustment tube 457.

Proximal end 452 is to be kept outside of the patient's body, and distal end 451 is configured to be placed in a blood vessel, during the revascularization procedure. Elongate tubular shaft 453 is the body of the PTVI device coupled between proximal end 452 and distal end 451. Lumen 454 extends within elongate tubular shaft 453 and includes a proximal opening 472 at proximal end 452 and a distal opening 471 at distal end 451. Lumen 454 is configured to accommodate at least a portion of the angioplasty catheter and allow the distal end of the angioplasty catheter to enter proximal opening 472 and exit from distal opening 471. In one embodiment, in which PTVI device 450 is an introducer sheath, lumen 454 is configured to accommodate at least a portion of the guide catheter and allow the distal end of the guide catheter to enter proximal opening 472 and exit from distal opening 471.

Adjustable balloon 456 is incorporated onto elongate tubular shaft 453 and configured to occlude a portion of a blood vessel when being inflated. Adjustable balloon 456 includes a chamber 459 having an adjustable volume. Balloon 456 is illustrated in FIGS. 4-8 in its inflated state. During insertion of system 110 into the femoral artery, balloon 456 is in its deflated state and wrapped tightly down onto elongate tubular shaft 453. In the deflated state, balloon 456 is a minimal addition to the diameter of elongate tubular shaft 453. In various embodiments, balloon 456 is soft and compliant to features of the artery wall to avoid disruption of the wall or disruption of arterial plaque. Balloon 456 is constructed to become fully inflated and occlusive at a minimal pressure above ambient blood pressure. Examples of suitable materials for constructing balloon 456 include silicone, latex, and polyurethanes. Elongate balloon adjustment tube 457 is coupled to adjustable balloon 456 and includes a fluid passageway 458 that is in fluid communication with chamber 459 to allow inflation and deflation of adjustable balloon 456. In one embodiment, a portion of elongate balloon adjustment tube 457 is affixed onto elongate tubular shaft 453. In another embodiment, a portion of fluid passageway 458 extends through a portion of elongate tubular shaft 453 as an additional lumen.

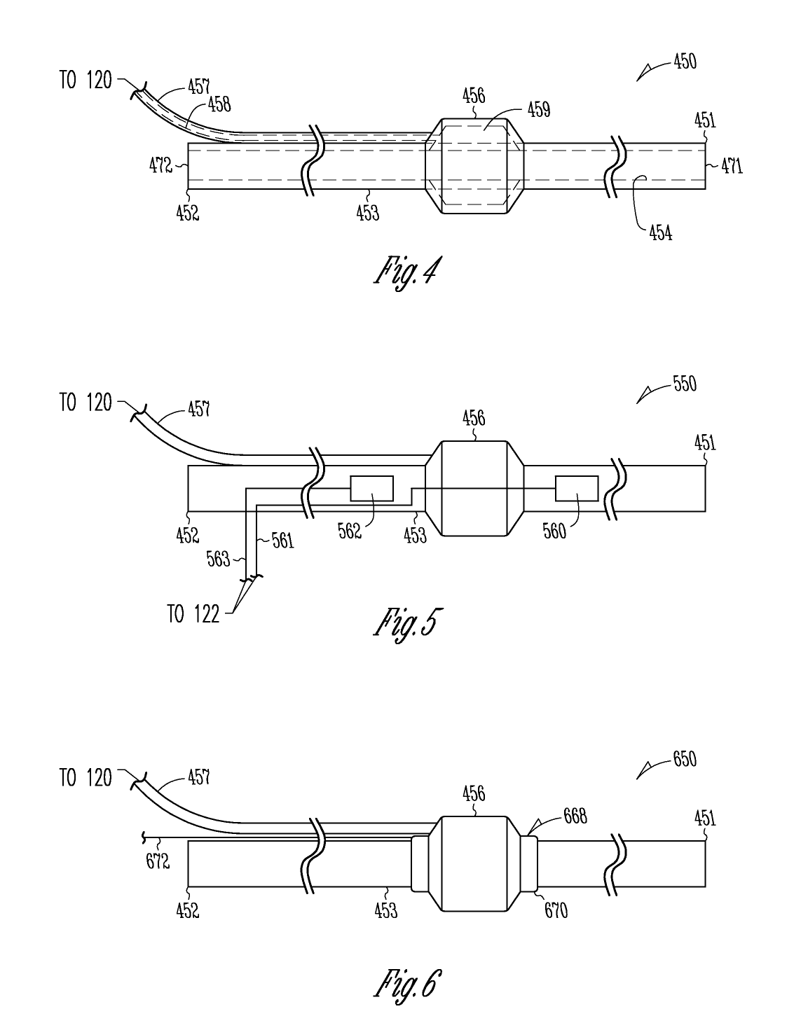

FIG. 5 is an illustration of an embodiment of a PTVI device 550 for remote ischemic conditioning. PTVI device 550 is part of PTVI device assembly 110 and includes the structure of PTVI device 450 and additionally a proximal sensor 562 and/or a distal sensor 560.

In various embodiments, proximal sensor 562 is incorporated onto elongate tubular shaft 453 in a location proximal to adjustable balloon 456, and includes one or more of a proximal flow sensor to monitor blood flow and a proximal pressure sensor to monitor blood pressure at a vascular location downstream to the occlusion site (wherein adjustable balloon 456 is placed). Distal sensor 560 is incorporated onto elongate tubular shaft 453 in a location distal to adjustable balloon 456, and includes one or more of a distal flow sensor to monitor blood flow and a distal pressure sensor to monitor blood pressure at a vascular location upstream to the occlusion site. In one embodiment, proximal sensor 562 includes the proximal flow sensor and the proximal pressure sensor, and distal sensor 560 includes the distal flow sensor. In various other embodiments, PTVI device 550 includes one or both of proximal sensor 562 and distal sensor 560, each of which include one or both of a flow sensor and a pressure sensor.

A lead 563 is connected to proximal sensor 562 to provide for connection between proximal sensor 562 and monitor 122, such that the sensed blood pressure is processed by monitor 122 for recording and/or presentation to the physician or other caregiver during the revascularization procedure. A lead 561 is connected to distal sensor 560 to provide for connection between distal sensor 560 and monitor 122, such that the sense blood flow or pressure is processed by monitor 122 for recording and/or presentation to the physician or other caregiver during the revascularization procedure.

FIG. 6 is an illustration of an embodiment of a PTVI device 650 for remote ischemic conditioning. PTVI device 650 is part of PTVI device assembly 110 and includes the structure of PTVI device 450 except that balloon 456 is made displaceable along a portion of elongate tubular shaft 453.

In the illustrated embodiment, PTVI device 650 includes a sliding balloon assembly 668 over elongate tubular shaft 453 and displaceable along a portion of elongate tubular shaft 453. Sliding balloon assembly 668 includes a sliding sleeve 670 over the portion of elongate tubular shaft 453, adjustable balloon 456 constructed on sleeve 670, a positioning member 672 to position sliding sleeve 670 and adjustable balloon 456, and elongate balloon adjustment tube 457. In various embodiments, positioning member 672 includes a wire, a tube, or another structure that allows for positioning of adjustable balloon 456 along the portion of elongate tubular shaft 453 from outside of the patient's body when adjustable balloon 456 is in the patient's body. In one embodiment, positioning member 672 is affixed to sliding sleeve 670. In one embodiment, a portion of elongate balloon adjustment tube 457 is integrated with positioning member 672. In another embodiment, elongate balloon adjustment tube is constructed, with sufficient stiffness, for positioning the adjustable balloon, eliminating the need for a separate positioning member.

FIGS. 4-6 illustrate various embodiments, by way of example and not by way of limitation, of a PTVI device including an adjustable balloon for remote ischemic conditioning during a PTCA procedure. In various other embodiments, one or more PTVI devices may be made by combining the features illustrated in FIGS. 4-6. For example, in one embodiment, proximal sensor 562 and distal sensor 560 are incorporated onto PTVI device 650, and sliding balloon assembly 668 is displaceable along elongate tubular shaft 453 between proximal sensor 562 and distal sensor 560.

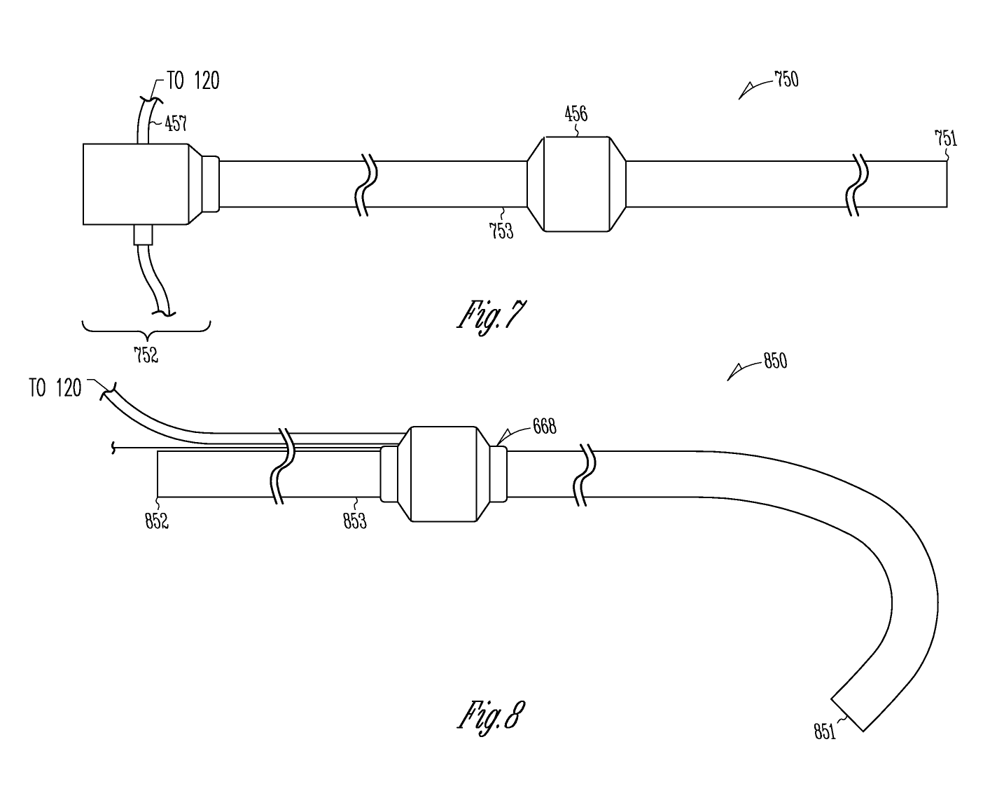

FIG. 7 is an illustration of an embodiment of an introducer sheath 750 with adjustable balloon 456 for remote ischemic conditioning. Introducer sheath 750 is an embodiment of PTVI device 450 or 550 being an introducer sheath. In one embodiment, adjustable balloon 456 is incorporated onto an introducer sheath platform being substantially similar to a Super Sheath™ introducer sheath (Boston Scientific Corporation).

Introducer sheath 750 includes a proximal end portion 752, a distal end 751 configured to be placed in the femoral artery, and an elongate tubular shaft 753. Elongate tubular shaft 753 has a length suitable for allowing a major portion of it to be placed within the femoral artery. In one embodiment, elongate tubular shaft 753 has a length between approximately 7 cm and 25 cm, with approximately 7 cm, 11 cm, and 25 cm being specific examples. In one embodiment, adjustable balloon 456 is affixed onto elongate tubular shaft 753 at a predetermined location.

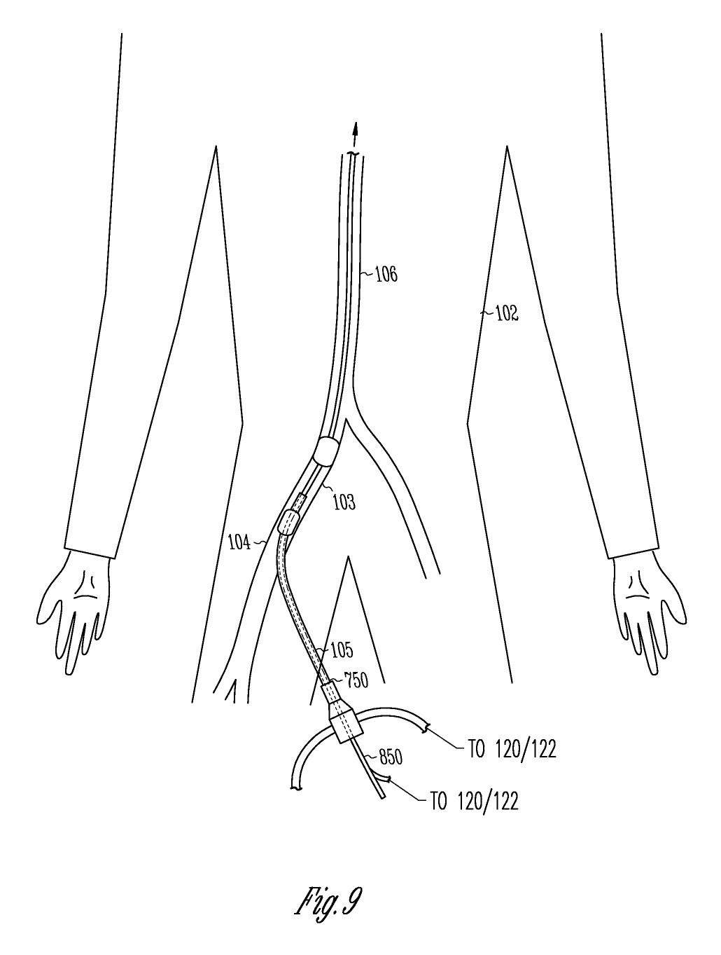

FIG. 8 is an illustration of an embodiment of a guide catheter 850 with sliding balloon assembly 668 for remote ischemic conditioning. Guide catheter 850 is an embodiment of PTVI device 650 being a guide catheter. In one embodiment, sliding balloon assembly 668 is incorporated onto a guide catheter platform being substantially similar to a guide catheter selected from the Runway®, Mach 1®, and Wiseguide® guide catheters (Boston Scientific Corporation).

Guide catheter 850 includes a proximal end 852, a distal end 851 configured to be placed in the aorta to engage the coronary artery, and an elongate tubular shaft 853. Elongate tubular shaft 853 has a length suitable for allowing distal end 851 to enter the femoral artery and advanced in the arteries to engage the coronary artery while proximal end 852 remains external to the patient's body. In one embodiment, elongate tubular shaft 853 has a length between approximately 90 cm and 130 cm, with approximately 100 cm being specific examples. Sliding balloon assembly 668 is displaceable along a portion of elongate tubular shaft 853.

In the embodiment, guide catheter 850 can slide longitudinally in the arteries relative to sliding balloon assembly 668 before and after adjustable balloon 456 is inflated. After inflation adjustable balloon 456, sliding balloon assembly 668 is fixed at a location in the descending aorta, iliac artery, or femoral artery. The physician or other caregiver operator may slide guide catheter 850 relative to the fixed position of sliding balloon assembly 668 in order to adjust the distal opening of guide catheter 850 relative to the coronary artery ostium as needed during the angioplasty procedure. The drop in blood pressure across the inflated balloon assembly 668 may require that the gap between guide catheter 850 and balloon assembly 668 to be small to prevent significant leakage of blood through this space. In one embodiment, sliding balloon assembly 668 includes a hemostasis seal to prevent such blood leakage.

FIG. 9 is an illustration of an embodiment using introducer sheath 750 and/or guide catheter 850 during a revascularization procedure. In one embodiment, one of introducer sheath 750 and guide catheter 850 is selected for applying the remote ischemic conditioning sequence during the revascularization procedure, depending on the desirable remote ischemic conditioning site. For example, introducer sheath 750 is selected to temporary occlude a portion of femoral artery 104, and guide catheter 850 is selected to temporary occlude a portion of iliac artery 103. In another example, guide catheter 850 is selected when the exact remote ischemic conditioning site may need to be determined during the revascularization procedure. In another example, guide catheter 850 is used for eliminating a potential need for multiple versions of catheters each with the adjustable balloon fixed at a predetermined location. In another embodiment, introducer sheath 750 and guide catheter 850 are both introduced into body 102 for additional flexibility during the revascularization procedure.

It is to be understood that the above detailed description is intended to be illustrative, and not restrictive. For example, the technique for applying remote ischemic conditioning as discussed in this document is not limited to use during revascularization, but may be used in vascular intervention procedures other than revascularization when performed on patients suffering from myocardial ischemia or infarction. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A percutaneous transluminal vascular intervention (PTVI) device for use with an angioplasty catheter during revascularization of a coronary artery in a body and for placement in one or more arteries through which a distal end of the angioplasty catheter is advanced into the coronary artery, the PTVI device comprising:
a proximal end;
a distal end configured to be placed in the one or more arteries;
an elongate tubular shaft coupled between the proximal end and the distal end,
a lumen extending within the elongate tubular shaft and including a proximal opening at the proximal end and distal opening at the distal end, the lumen configured to accommodate a portion of the angioplasty catheter and allow the distal end of the angioplasty catheter to enter the proximal opening and exit from the distal opening;
an adjustable balloon incorporated onto the elongate tubular shaft and configured to occlude a portion of the one or more arteries, the balloon including a chamber having an adjustable volume; and
an elongate balloon adjustment tube coupled to the adjustable balloon and including a fluid passageway in fluid communication with the chamber to allow inflation and deflation of the adjustable balloon.

2. The PTVI device of claim 1, comprising a distal flow sensor incorporated onto the elongate tubular shaft in a location between the distal end and the adjustable balloon to monitor blood flow.

3. The PTVI device of claim 2, comprising a proximal pressure sensor incorporated onto the elongate tubular shaft in a location between the proximal end and the adjustable balloon to monitor blood pressure.

4. The PTVI device of claim 2, comprising a proximal flow sensor incorporated onto the elongate tubular shaft in a location between the proximal end and the adjustable balloon to monitor blood flow.

5. The PTVI device of claim 1, comprising a sliding balloon assembly over the elongate tubular shaft, the sliding balloon assembly displaceable along a portion of the elongate tubular shaft and including the adjustable balloon.

6. The PTVI device of claim 5, wherein the sliding balloon assembly comprises a positioning member adapted to position the adjustable balloon.

7. The PTVI device of claim 6, wherein a portion of the elongate balloon adjustment tube comprises the positioning member.

8. The PTVI device of claim 6, wherein the sliding balloon assembly comprises a sliding sleeve over the portion of the elongate tubular shaft, the adjustable balloon is constructed on the sleeve, and the positioning member is affixed to the sliding sleeve.

9. The PTVI device of claim 1, comprising an introducer sheath including the proximal end, the distal end, the elongate tubular shaft, the adjustable balloon, and the elongate balloon adjustment tube.

10. The PTVI device of claim 9, wherein the elongate tubular shaft has a length between approximately 7 cm and 25 cm, and the adjustable balloon is affixed onto the elongate tubular shaft at a predetermined location of the elongate tubular shaft.

11. The PTVI device of claim 1, comprising a guide catheter including the proximal end, the distal end, the elongate tubular shaft, the adjustable balloon, and the elongate balloon adjustment tube.

12. The PTVI device of claim 11, wherein the elongate tubular shaft has a length between approximately 90 cm and 130 cm, and a sliding balloon assembly over the elongate tubular shaft, the sliding balloon assembly displaceable along a portion of the elongate tubular shaft and including the adjustable balloon.

13. A method for cardioprotection during revascularization of a coronary artery, the method comprising:
inserting a portion of a percutaneous transluminal vascular intervention (PTVI) device into one or more arteries, the PVTI device including an adjustable balloon;
inserting a portion of an angioplasty catheter into a lumen of the PTVI device;
advancing a distal end of the angioplasty catheter through the PTVI device into the coronary artery; and
occluding a portion of the one or more arteries temporarily by inflating and deflating the adjustable balloon, the occluding timed using parameters selected to create a physiologic stress that is sufficient to trigger an intrinsic myocardial protective mechanism.

14. The method of claim 13, wherein occluding a portion of the one or more arteries temporarily comprises applying an ischemic conditioning sequence including a specified number of cycles of alternating ischemic and non-ischemic periods, the ischemic periods each including an ischemic duration during which the adjustable balloon is inflated to temporarily occlude the portion of the one or more arteries, the ischemic periods each including a non-ischemic duration during which the one or more balloons are deflated.

15. The method of claim 14, comprising programming the number of cycles to a number in a range of approximately 1 cycle to 10 cycles, programming the ischemic period to a time interval in a range of approximately 1 minute to 20 minutes, and programming the non-ischemic period to a time interval in a range of approximately 1 minute to 10 minutes.

16. The method of claim 14, wherein inserting the portion of the PTVI device into the one or more arteries comprising inserting a portion of an introducer sheath into a femoral artery such that the adjustable balloon is placed in the femoral artery.

17. The method of claim 14, wherein the adjustable balloon is displaceable along a portion of the PTVI device, and comprising sliding the adjustable balloon relative to the PTVI device to the portion of the one or more arteries to occlude the portion of the one or more arteries.

18. The method of claim 17, wherein inserting the portion of the PTVI device into the one or more arteries comprising inserting a portion of a guide catheter into the one or more arteries, and sliding the adjustable balloon comprises sliding the adjustable balloon to an iliac artery or a descending aorta.

19. The method of claim 13, comprising sensing blood flow or blood pressure at a vascular location downstream to the portion of the one or more arteries using one or more sensors incorporated onto the PTVI device.

20. The method of claim 13, comprising sensing blood flow or blood pressure at a vascular location upstream to the portion of the one or more arteries using one or more sensors incorporated onto the PTVI device.
